(19) United States
(12) Patent Application Publication
(10) Pub. No.: US 2007/0179362 A1
(43) Pub. Date: Aug. 2, 2007

(54) Method of feedbacking physical condition of fetus and gravida automatically

(76) Inventor: Chun-Mei Chou, Jhunan Township (TW)

Correspondence Address: HDSL, 4331 STEVENS BATTLE LANE, FAIRFAX, VA, 22033, US

(21) Appl. No.: 11/341,472

(22) Filed: Jan. 30, 2006

(51) Int. Cl. A61B 5/00; A61N 1/00; A61B 5/04; A61B 8/00; A61B 5/103

(52) U.S. Cl. 600/301; 600/546; 600/376; 600/453; 600/511; 600/588; 607/46; 128/905

(57) ABSTRACT

A method and an instrument feed back the physical condition monitored from the fetus and the gravida automatically. Various monitoring methods are adopted to automatically set up monitoring intervals and time points. While the physical condition detected is good, it keeps the preset monitoring time intervals; if the feedback condition is not good, then the monitoring intervals and time points would be adjusted, and the instrument informs the gravida for further management. Through that, it generalizes all the values monitored at different time. The detailed result could be got from feedback of the repeated monitoring and cross monitoring thereby automatically, then well informing the gravida about the condition of herself and her fetus to get proper medical assistance.

### BACKGROUND OF THE INVENTION

1. Field of the Invention

This invention contains a method and a device of feeding back the physical condition of a fetus and a gravida automatically, especially directs to a device installed on an attire, using the theory of fetal heart sound monitoring, accompanying with different automatically monitoring methods such as EMG, EKG, ultrasound image, and stress monitoring, blood pressure, heart beat and so on; and automatically feeding back the repeated monitored values to provide the latest physical condition of a gravida and her fetus to remote monitoring center for offering medical assistance when it is necessary.

2. Description of the Prior Art

It's joyful to welcome a baby's birth. Especially with the expectation and blessing of everyone in the family, the moment of baby's birth sheds the light of happiness.

However, it's known to everyone that the gravida and her fetus are fragile. Though modern medicine has been improved a lot ever since, it still cannot resolve certain issues about life creation in advance.

Therefore, it is necessary to observe and follow up in a long run for the health care of the gravida and her fetus.

The major conventional way of monitoring the fetus is Not Stress Test (NTS), wherein by using the uterus contraction meter to continuously monitor fetus' heart beat and uterus contraction of the gravida, and the gravida presses the recording button once there is a fetal movement. Through studying the record of fetal movement, the result is shown below:
A. Reactive: When there is more than two fetal movements in 20 minutes, and the fetus's heart beat is above 15 times per sec which remains at least 15 seconds; this is regarded as normal. The condition should be without variable deceleration and kept for 7 days.
B. Non-Reactive: wherein the result is out of the range of reactive mentioned previously, then the fetus might be sleeping, we could ask the gravida to eat something sweet or be given glucose iv fluid, and then laying left lateral or half laying lateral and keep recording; if there is still non-reactive, then the possibility of fetal distress increases. The term “fetal distress” indicates that the fetus is under low blood oxygen, high blood carbon dioxide, or metabolic acidosis. If proper process is not taken immediately, the central nervous system or other organs of the fetus might be damaged, or fetal death might occur. The cases that often cause fetal distress such as cord compression, strong uterus contraction strong, placenta malfunction, umbilical cord around the neonatal neck or oligohydramnios. The way to resolve this issue is to give oxygen to the gravida, iv fluid supplement, or let the gravida lying lateral; if the condition is not improved, then the doctor will let the gravida giving birth immediately. If the gravida cannot give birth naturally in a short time, then it's necessary to proceed to Ceaser's section to save the mother and her baby.

The monitoring method above is processed once a week when it's pregnant for 36 weeks. But for high risk gravida who has high blood pressure, diabetes, overdue, uterus growth delayed, the history of miscarriage, anemia, hemolytic disease, inheritance disease, heart disease, fetus too small, less fetal movement, collagen disease, preterm birth, premature rupture of membranes or the other reasons that is under bad condition judged by doctor, then it's suggested to practice twice every week after the 32nd week of pregnant.

However, even the conventional monitoring ways are administrated, there are still serious medical dispute happened for the cases that fetus is normal under NST monitoring but the newborn died in seven days after birth. Hence, the methods mentioned in this invention provide important information automatically to the gravida and the monitoring center to help define the responsibility. It also provides the gravida safety monitoring method, and information of clear fetus heart beat, fetal movement, position or the edema of the gravida and rupture of membranes of the gravida etc.; the invention offers continuous monitoring to provide evidence when there is a dispute.

Another report indicates that through continuously using fetus heart monitoring device, an unacceptable result displayed always makes the doctors and the gravida nervous, and decides processing Ceaser's section, which make this procedure rank high in the area that uses fetus heart monitoring device often. This is not a good way to practice medicine. For that reason, this invention provides correct fetus condition to monitoring center; no matter it's rupture of membranes, abnormal uterus contraction, less fetal movement or abnormal heart beat, fetal malpresentation, or edema of the gravida, high blood pressure and so on, are all recorded for providing correct information to the monitoring center for making proper decision.

Based on the defects found on the conventional methods, the inventor invents this interruptible monitoring method, enables the gravida with various conditions receiving proper medical help, and improves the health of the fetus and the gravida. The invention could be wear on and without interfering daily life of the gravida, thus reduce the mal-effect on the gravida.

### SUMMARY OF THE INVENTION

This invention is to offer a method, which automatically feeds back the physical condition of the fetus and the gravida. Through the signals of the three EMG wearing on three different quadrants of the gravida, the uterus contraction and fetal movement could be recorded. If the uterus contraction is happened after the 36^{th} weeks of pregnant, this could be regarded as normal; otherwise the gravida needs to receive tocolysis. If the gravida has serious edema recently, then she should be given health education and high protein diet to improve the health condition of the gravida and the fetus.

Further, this invention is to provide a method that automatically feeds back the physical condition of the fetus and the gravida, wherein the ultrasound image localization is used to measure the EKG or heart sound of the fetus immediately. The other organs' sound might interfere fetal heart sound and cause false positive when examines by conventional ways. Therefore, the ultrasound image localization used here is to avoid this error and prevent tragedy from happening.

Another, this invention is to provide a method that automatically feeds back the physical condition of the fetus and the gravida. According to the real experience of the inventor, and back to value the importance of fetal movement. Making good use of EMG monitoring on different quadrants of the fetus to correctly evaluate the fetal movement and development of the fetus, thus make this invention become an objective and correct indicator for health evaluation.

This invention uses a new way of cross monitoring method to evaluate the growth and heart beat of the fetus and the blood pressure, edema, heart beat and body temperature of the gravida. Wherein the various monitors are put on the abdominal of the gravida and the doctor judge the cycle and time to monitor, e.g. monitoring the fetus by fetal heart beat monitor with the time intervals described previously, if the result is good, then continues the monitoring according to the time interval set previously. If the monitored result is not good, then increases the monitoring cycle and time points, and ask the physician for help. At the same time, the ultrasound image monitor, EKG monitor, pressure gauge, sphygmomanometer, or the other proper monitoring methods are used at different point and feedback repeating monitoring, crossing-monitoring automatically, and then generalized all the values monitored and got the detailed monitored result, which is provided to the doctor for giving proper medical assistance to the gravida.

### BRIEF DESCRIPTION OF THE DRAWINGS

FIG. 1 is the implementation of this invention.

FIG. 2 displays how to wear the monitoring device of this invention.

FIG. 3 displays the set up flow chart of this invention.

FIG. 4 displays the flow chart of the embodiment of this invention.

### DETAILED DESCRIPTION OF THE PREFERRED EMBODIMENT

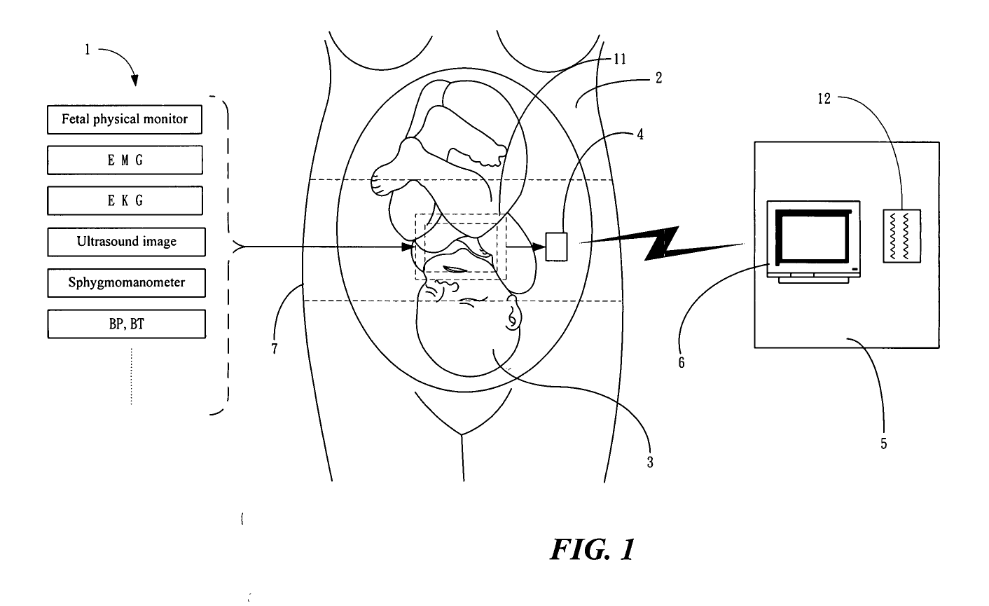

As shown in FIG. 1, a device which automatically feedbacks the physical condition of the fetus and the gravida includes a monitoring device 1. The monitoring device 1 includes a fetal physical monitor 11, EMG, EKG, ultrasound, ultrasound image, pressure gauge, device that monitors the gravida's blood pressure, heart beat, edema, body temperature or the others. Install the monitoring device 1 on the attire 7 dressed by the pregnant woman 2, and then use it to monitor physical condition of the gravida 2 and the fetus 3. The signal monitored by the monitoring device 1 is transmitted to monitoring center for further analysis, record and display, and then feed back the result to monitoring device 1 and continue the monitoring, and transmitted to remote monitoring center for analysis by wireless, telephone, cellular phone, PDA and network or let the doctor judge and decides whether to provide medical aids according to the signals received. The gravida herself can interact with medical staff at remote site or check out the monitoring result. Medical staff at remote site could use video or voice devices to process conversation.

The monitoring cycle and time interval of monitor 1 could be adjusted. As the technology described previously, the gravida 2 is administered continuous or multiple monitoring process with fetal physical monitor 11 at 37 weeks of pregnant; if there is more than twice fetal movement in 20 minutes, the fetal heart beat rate more than 15 times per minute, and the condition keeps at least 15 seconds every time, this is regarded as normal; wherein the setting up criteria is depended on the physical condition of the user, otherwise there needs another process. There is a displayer 6 at remote monitoring center 5, at which a uterus contraction displayer 12 or the other monitors 11 described previously could be installed to display result for the doctor making diagnosis.

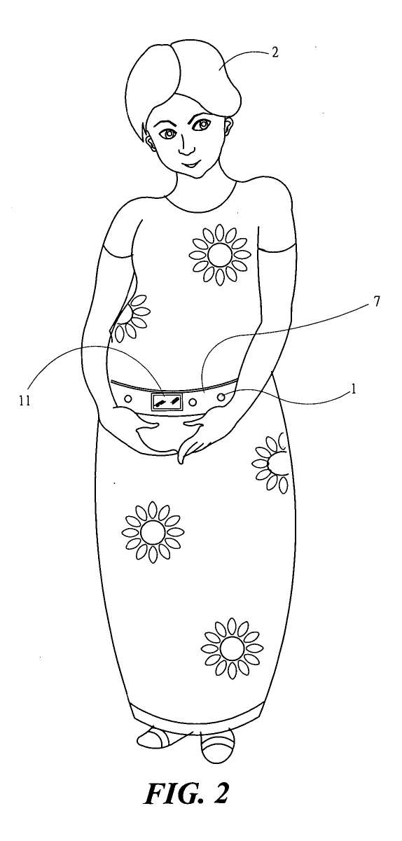

As displayed in FIG. 2, the monitor 1 of the invention is installed on the attire 7 (e.g. the underwear or abdominal binder) for convenience. By wearing these devices on to fix them on the body of the gravida, if there is no signal detected, the device can make sound to signal the gravida to adjust the position or the monitor 1 can automatically adjust itself to proper position, the gravida remains her daily life without being interfered. The monitoring time is automatically set up by the monitor 1, or could be set up/adjusted automatically by near-end monitoring center 4 or by remote monitoring center 5. The monitored result is directly displayed on the attire 7 such as the fetus physical monitor 11 with image displayer described previously, which enables the gravida to monitor the physical condition of her fetus and herself anytime.

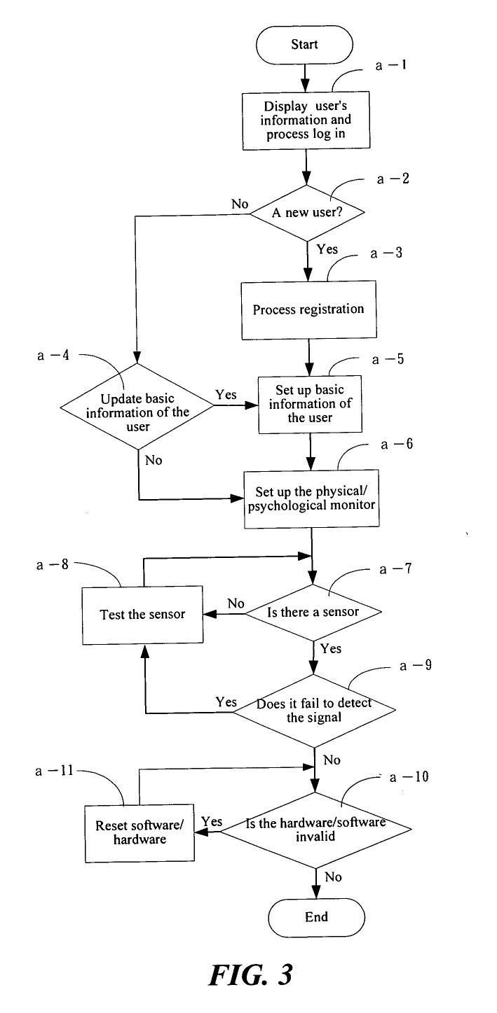

As shown in FIG. 3, there are some settings of the invention need to be done in advance, the procedure is listed below:
a-1 the system displays log in information for user to proceed to next step;
a-2 the system decides if this is a new user;
a-3 if this is a new user, the system prompts user to register;
a-4 if the user is already registered, then the system asks user if she would like to update her data;
a-5 The system set up user's basic information such as height, weight, age, medical history and so on, then the system set up the monitoring time and interval based on this basic information.
a-6 Set up the monitoring devices;
a-7 Examine if there is a sensor: if there is no response, then proceeds to a-8; otherwise proceeds to a-9.
a-8 Check the sensor again;
a-9 Examine whether the sensor works: if the sensor is not working, check it up again.
a-10 Check up whether the other hardware/software is valid, if any one of them is invalid, then proceeds to a-11.
a-11 Set up the hardware/software.
   By practice the procedures described above to enable the monitoring system of the invention.

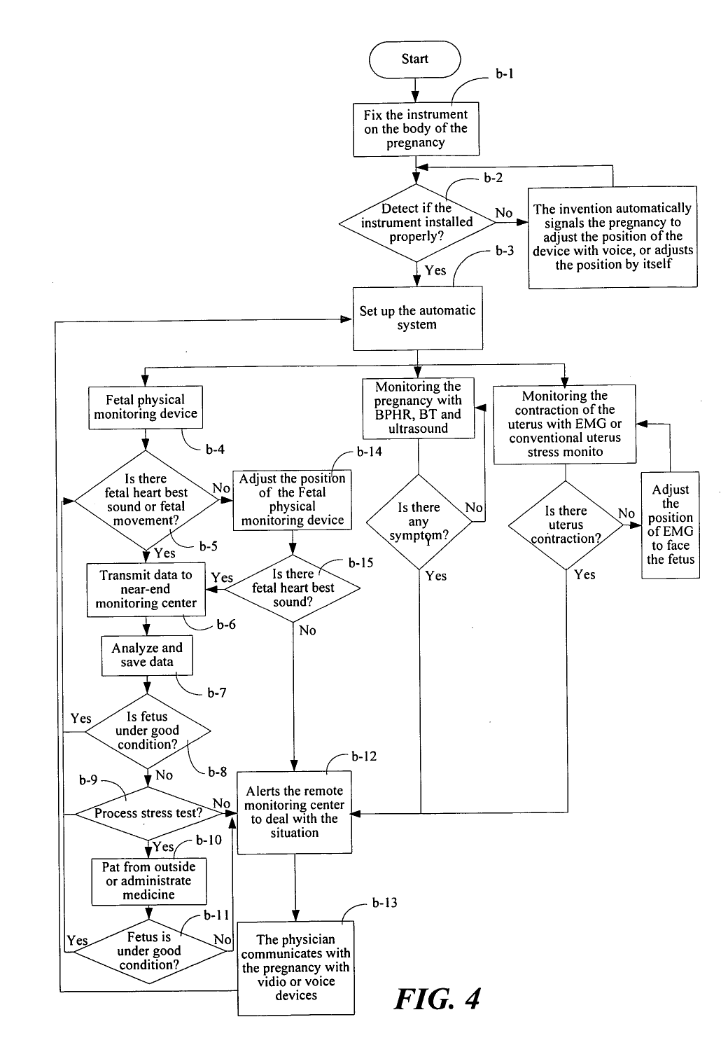

As shown in FIG. 4, the monitoring procedure of the invention is described as below:
b-1 The gravida 2 puts on the attire 7 on which the monitor device 1 is installed. The proper monitoring device 1 (e.g. fetal physical monitor 11) is turn on to process testing;
b-2 Examine if the monitoring device 1 has signal, the device can make sound to indicate the gravida to adjust the position or the monitor 1 can automatically adjust itself to proper position, until the signal of the fetus is detected;
b-3 The monitoring setting that is set up according to the basic information input by the gravida could be automatically adjusted if an abnormal condition is detected;
b-4 Choose the monitoring items, such as fetal heart sound, fetal movement, uterus contraction and so on. Using fetal heart sound as an example:
b-5 The invention detects fetal heart sound, the status of fetal movement, and the physical condition of the gravida such as heart rate, blood sugar, and blood pressure and so on.
b-6 If there are fetal heart sound and fetal heart beat, the signal would be continuously transmitted to near-end monitoring center 4;
b-7 Data analysis and storage;
b-8 Decide if the fetus 3 or the gravida is under good condition, if the answer is yes, then the invention continuously monitors and feedbacks monitoring repeatedly.
b-9 If the fetus or the gravida is under bad condition, then decides whether to process stress test;
b-10 The gravida is given stress test such as pat from outside of the abdominal or given medicine (e.g. uterus contraction agents).
b-11 Decide if the fetus 3 or the gravida is under good condition, if the answer is yes, then continuously monitor and feed back repeated monitoring.
b-12 If the fetus 3 or the gravida is under bad condition, or the gravida is not given stress test; then signals the gravida and continues monitoring. If the fetus 3 or the gravida physical condition is not improved, then the signal is transmitted to remote monitoring center 5 for further diagnosis.
b-13 The remote monitoring center 5 give indication based on the information got. The remote monitoring center 5 contacts the gravida 2 at time indicated or any time to provide the gravida 2 the preferred indication.
b-14 If step e cannot detect fetal heart sound, it firstly decide if the position is proper, if it is not correct, then adjusts fetal physical monitor 11.
b-15 Detect whether there is fetal heart sound, if it has then proceeds to step f; if no heart sound detected, then proceeds to step i emergency process.

Besides, the ultrasound device mentioned above could be used to monitor heartbeat of the fetus 3, malformation, fetal movement, fetal growth abnormality, or rupture of the membranes, and any change in heart, liver, cholocyst and so on of the gravida. Wherein the monitoring device 1 could be ultrasound, sound monitoring device, stress monitoring device, EMG, EKG, BP, HR, and BT. For example: ultrasound, EMG, and sound monitoring device could be used to monitor fetal heart sound; EMG, BP, HR, and BT could be used to monitor the physical condition of the gravida 2. Through cross-monitored by various monitoring device 1, we could get complete monitored values. The monitoring device 1 could be implemented into the body of the gravida 2 when it is necessary, e.g. the secretion of hormone, prolactin, or the amount of blood sugar, endocrine and so on could be measured by biochips.

The hormone kits mentioned above comprises β-HCG, progesterone, oxytocin, prolactin, FSH, LH, TSH, T4, T3, E2 and so on, wherein β-HCG and E2 could be used to test if the woman is pregnant; the physician could examine the amount of progesterone, oxytocin, and prolactin to judge the condition of the gravida. It provides a good way to monitor the gravida by taking good use of these hormone kits and installing biochips in tampon. Extensively, the invention could provide its monitoring function to general people; and by the monitoring method mentioned previously, the necessary therapies such as transcutaneous electrical nerve stimulation (TENS) could be administrated, ultrasound therapy, pat therapy and injection therapy and so on. TENS could be administrated by using EMG, and ultrasound itself could be used in monitoring and therapy, so it is not necessary to add extra device for this invention to provide monitoring and therapy functions.

The monitoring device 1 described above could be the composition of three or more monitors, e.g. the fetal heart sound monitor is composed by ultrasound device, sound monitor device and EKG; fetal movement monitor is composed by ultrasound or stress monitoring device; uterus contraction monitor could comprise EMG or traditional uterus contraction monitor. The invention could inform (or alert), communicate and the gravida 2 by cross-judgment through various monitoring devices 1, and communicate or process correction whenever it is necessary. The invention would transmit signal to remote monitoring center 5 and build communication between medical staff and the gravida 2, in order to give indication for further physical testing or how to do next.

Furthermore, the invention could be used to examine the physical condition of the gravida 2 in advance based on the values detected by various monitoring devices; and the condition could be deal with as early as possible. For example:
A. The amount of amniotic fluid could be measured by ultrasound, once the rupture of membranes, the invention will inform the gravida 2, and the gravida 2 inform remote monitoring center 5 for further process. If the physical condition of the gravida 2 is found abnormal, the device informs the gravida for immediate process or let the physician at remote monitoring center give medical order.
B. The amount of blood sugar of the gravida 2 could be detected by the blood sugar monitoring device or biochip. If it is above normal value, then the near-end monitoring center 4 automatically alerts the gravida 2 and let the gravida 2 notice the control of blood sugar, the information also transmit to remote monitoring center 5 for providing medical assistance.
C. The blood pressure of the gravida 2 could be detected, if it is above normal value, then the near-end monitoring center 4 remind the gravida to improve her diet, and control the intake of salt, water and take medicine on time.
D. EMG could detect the condition of edema. Once edema is detected, the device informs the gravida to limit the intake of salt and water.

The recovery of the gravida 2 from high blood pressure and edema mentioned above should be attended anytime. If the physical condition of the gravida 2 is not improved and the gravida is less than 36 weeks, then there should be seriously monitor and control the physical condition of the gravida 2 and the fetus 3. If the pregnant is over 36 weeks, then the remote monitoring center 5 informs medical staff to evaluate the condition and prepare for the possibility of delivery.

From the description above, both the near-end monitoring center 4 and remote monitoring center 5 process the cross detection and mutual communication, which makes the gravida 2 is able to be well informed and manage her physical condition, and makes it possible for the gravida 2 not to stay in delivery center for testing; and the medical staff could get the testing result of the gravida 2, communicate with the gravida 2, be notified about the physical condition of the gravida 2, and give suggestion to the gravida 2 anytime.

What is claimed is:

1. A method which feeds back physical condition of a fetus and a gravida automatically, comprising the steps of:
disposing on a surface of the gravida with three or more EMG devices at different quadrants and get condition of uterus contraction, fetal movement and edema of the gravida, in order to evaluate the physical condition of the fetus and the gravida and give medical assistance whenever it is needed; and
by using a ultrasound image localization device to administrate fetal EKG, fetal heart sound monitoring and fetal movement monitoring, which actually displays the fetal heart beat rhythm and condition of the fetal movement at real time, and avoids a false positive.

2. The method as recited in claim 1, further comprising monitoring stress, edema of the gravida, heart beat, blood pressure and body temperature.

3. The method as recited in claim 1, further comprising judging whether the EMG or the ultrasound image localization device is out of order or is put on wrong position, or the fetus' position is changed, and then processes necessary adjustment when no signal or weak signal is detected.

4. The method as recited in claim 1, wherein the EKG or the ultrasound image localization device are installed on the attire, and by wearing the attire to secure on the surface of the body of the gravida.

5. The method as recited in claim 1, wherein the EMG provides the information about fetal movement, edema or uterus contraction of the gravida, and proceeds to transcutaneous electrical nerve stimulation (TENS) to reduce or comfort back sore of the gravida.
